(19) United States
(12) Patent Application Publication
(10) Pub. No.: US 2023/0201625 A1
(43) Pub. Date: Jun. 29, 2023

(54) SYSTEM AND METHOD FOR LASER BASED WHITENING TREATMENT OF HARD TISSUE

(71) Applicant: Convergent Dental, Inc., Needham, MA (US)

(72) Inventor: Charles Kerbage, Arlington, MA (US)

(21) Appl. No.: 18/089,372

(22) Filed: Dec. 27, 2022

Related U.S. Application Data
(60) Provisional application No. 63/294,011, filed on Dec. 27, 2021.

(51) Int. Cl. A61N 5/06; A61C 19/06; A61N 5/067; A61K 8/22; A61Q 11/00

(52) U.S. Cl. CPC A61N 5/062; A61C 19/066; A61N 5/0603; A61N 5/067; A61K 8/22; A61Q 11/00; A61N 2005/0606; A61N 2005/066; G02B 26/105

(57) ABSTRACT

The disclosed invention relates to a system and method for treatment of a dental hard tissue. The system can include a laser source for generating a laser beam; at least one optic in optical communication with the laser source and adapted to define and direct a non-ablative laser radiation to a treatment surface of the hard dental tissue; and a conduit adapted to apply a whitening treatment to the treatment surface, wherein application of both the non-ablative laser radiation and the whitening treatment results in a ΔE measurement of at least 2.5 times greater than a treatment surface without application of the non-ablative laser radiation.

### CROSS-REFERENCE TO RELATED APPLICATIONS

This application claims the benefit of priority to U.S. Provisional Patent Application No. 63/294,011 entitled “System and Method for Laser Based Treatment of Hard Tissue,” filed on Dec. 27, 2021, the contents of which are incorporated by reference herein in their entirety.

### FIELD OF THE INVENTION

This invention generally relates to laser-based treatment of hard tissue and more particularly to using lasers to enhance whitening, removal of stains, and strengthening of hard tissue by directing radiation emitted by a laser source to a hard tissue and application of a bleach solution.

### BACKGROUND

The mineral phase of human teeth comprises calcium phosphate in the form of hydroxyapatite, Ca₅(PO₄)₃(OH). Enamel, the outer part of a tooth, is a highly mineralized tissue containing about 97% hydroxyapatite. The enamel surface itself is covered by the pellicle, which contains mainly salivary proteins, carbohydrates, and lipids. The original color of pure hydroxyapatite (i.e., without substituting foreign ions) is colorless/white, which also broadly holds for the integrated proteins. Consequently, natural enamel has a white color with some translucency. The “natural” white color of teeth is often compromised due to stains resulting from wine, tea, coffee, smoking, etc. Whitening formulations for home and professional use in the dental practice increase the visual whiteness of a tooth. The in-office treatments in particular, often involve enamel etching or light.

In a typical treatment process, the dental bleaching material is brought into contact with the teeth. The bleaching effect depends on the duration of the time of the bleach on the surface of the tooth, the concentration of the peroxide and on the rate of bleach activation. The dental bleach is then allowed to remain in contact with the teeth for a certain time duration ranging from 10 minutes to one hour. Frequently used in-office and aggressive bleaching using peroxides is effective, but side effects like tooth sensitivity or a damage of the natural organic matrix of enamel and dentin may occur. In addition, mechanical weakening of the tooth due to a decreasing integration of the calcium phosphate crystals.

Both heat and light activation were demonstrated to increase the rate of bleaching of the hydrogen peroxide and providing a shorter period of time in which whitening of the teeth. The use of lasers to increase the rate of activation of the gel was first reported in 1918 by Abbot and hence enhance whitening effect by the raising the temperature of hydrogen peroxide and accelerates the rate of chemical bleaching of teeth.

One disadvantage of laser light enhancing methods is an undesirable heating of the hard tissue and of the pulp, possibly leading to irreversible damage and increased sensitivity. In particular, lasers such as Er:YAG and Nd:YAG that are primarily absorbed by water rather than hydroxyapatite, and subsequently may cause undesired damage to the surface of the tooth in the process of heating it. Addionally, significant amount of the water in the gel is heated before any acceleration of hydrogen peroxide diffusivity happens and renders breaks the gel and ultiamtley the technique inefficient.

CO₂-laser operating at the wavelength of 9.3 μm has been demonstrated by Featherstone and co-workers to enhance caries resistance by modifying enamel surface. In particular, dental enamel absorbs the CO₂-laser wavelengths 9.3 μm a factor of ten more than the conventional 10.6-μm CO₂-laser wavelength. The controlled heating removes the carbonate impurities from enamel mineral, driving it off as carbon dioxide and leaving behind mineral that is close in composition to pure hydroxyapatite. In addition, the high absorption coefficient of hydroxyapatite at 9.3 um wavelength, provides a shallow depth effect of the laser irradiation and also creates microscopic changes on the surface without jeopardizing of the sturcutre of tooth or increase the pulp temperature. As such, laser irradiation dental enamel that results in the removal of the carbonate also improves the absorption of exogenous material applied on the tooth. It has been demonstrated in multiple studies that applying fluoride to the tooth after laser irradiaiotn resulting in a further modified enamel surface that approaches the composition of the less soluble form of calcium phosphate known as fluorapatite, which is even more resistant to acid attack than hydroxyapatite. As such application of a gel that includes hydrogen peroxide and or fluoride for example would increase the hardness of the teeth and prevents from caries formation and also can enhance whitening.

Thus, there remain a need for a laser system adapted to treat hard tissue without damaging the hard tissue.

### SUMMARY

Accordingly, the present disclosure relates to a laser based treatment adapted for treating hard tissue. Example treament performed by the system can include using lasers to enhance whitening, removal of stains, and strengthening of hard tissue by directing radiation emitted by a laser source to a hard tissue and application of a bleach solution. Various embodiments of the system include an improved laser based device for treatment for inhibiting caries development and increased resitance to acid dissolution. For example, a CO₂ laser source can be used to operate in the range of 9-11 μm wavelength. CO₂ Lasers can have several advantages over hard tissue lasers (e.g., Er:YAG lasers) such as achieveing absorption coefficient of about 10 factors higher.

In various embodiments, the system includes a handpiece for directing radiation (e.g., a laser beam) in the near- to far-infrared spectra (e.g., 9-11 μm wavelength range), to allow for treatment of hard tissue in the oral cavity with optimal efficiency, minimal technique sensitivity, and a fast treatment time.

In some embodiments, the system can be adapted to scan the laser beam using known scanning technique, e.g., galvo-mirrors. The laser beam can be scanned across the treatment region using particular pattern(s) to allow for efficient energy delivery, producing enough localized photothermal effect to heat without damaging (burning or charring) of the tissue.

In some embodiments, the system may also include a laser source controller to adjust one or more parameters of the radiation (e.g., laser pulse duration) according to the type of treatment selected and/or the type of tissue being treated. For example, during treatment, the laser beam may be directed to the treatment area, allowing for delivery of a specified energy profile at or near the treatment area.

In one aspect, the invention relates to a system for treating a dental hard tissue. The system can include a laser source for generating a laser beam; at least one optic in optical communication with the laser source and adapted to define and direct a non-ablative laser radiation to a treatment surface of the hard dental tissue; and a conduit adapted to apply a whitening treatment to the treatment surface, wherein application of both the non-ablative laser radiation and the whitening treatment results in a ΔE measurement of at least 2.5 times greater than a treatment surface without application of the non-ablative laser radiation.

In some embodiments of the above aspect, the laser beam can include a wavelength in a range from 9 μm to 11 μm. In some instances, at least one optic can include a galvanometer and/or a turning mirror.

In some embodiments, the non-ablative laser radiation can include a fluence profile at a focus having: a maximum local fluence less than an upper threshold fluence, the upper threshold fluence defined as a minimum fluence that causes a surface modification of the dental hard tissue, and at least one other local fluence greater than a lower threshold fluence, the lower threshold fluence defined as a fluence that causes at least one of (i) acid dissolution and (ii) decrease in an amount of surface carbonate. In some instances, the surface modification can include melting and/or ablation.

In some embodiments, the whitening treatment can include bleach and/or hydrogen peroxide. In some instances, application of both the non-ablative laser radiation and the whitening treatment further results in a microhardness measurement of at least 18%, an inhibited demineralization of at least 64%, and/or a Vita shade guide measurement of at least 6 times greater than the treatment surface without application of the non-ablative laser radiation.

In another aspect, the invention relates to a system for treating a dental hard tissue. The system can include a laser source for generating a laser beam; at least one optic in optical communication with the laser source and adapted to define and direct a non-ablative laser radiation to a treatment surface of the hard dental tissue; and a conduit adapted to apply a whitening treatment to the treatment surface, wherein application of both the non-ablative laser radiation and the whitening treatment results in a microhardness measurement of at least 18% greater than a treatment surface without application of the non-ablative laser radiation.

In another aspect, the invention relates to a system for treating a dental hard tissue. The system can include a laser source for generating a laser beam; at least one optic in optical communication with the laser source and adapted to define and direct a non-ablative laser radiation to a treatment surface of the hard dental tissue; and a conduit adapted to apply a whitening treatment to the treatment surface, wherein application of both the non-ablative laser radiation and the whitening treatment results in a Vita shade guide measurement of at least 6 times greater than a treatment surface without application of the non-ablative laser radiation and the whitening treatment.

In another aspect, the invention relates to a method for treating a dental hard tissue. The method can include the steps of: generating a laser beam using a laser source; applying a non-ablative laser radiation to a treatment surface of the hard dental tissue using an optic in optical communication with the laser source; and after the step of directing the non-ablative laser radiation, applying a whitening treatment to the hard dental tissue, wherein application of both the non-ablative laser radiation and the whitening treatment results in a ΔE measurement of at least 2.5 times greater than a treatment surface without application of the non-ablative laser radiation and the whitening treatment.

In another aspect, the invention relates to a method for treating a dental hard tissue. The method can include the steps of: generating a laser beam using a laser source; applying a non-ablative laser radiation to a treatment surface of the hard dental tissue using an optic in optical communication with the laser source; and after the step of directing the non-ablative laser radiation, applying a whitening treatment to the hard dental tissue, wherein application of both the non-ablative laser radiation and the whitening treatment results in a microhardness measurement of at least 18% greater than a treatment surface without application of the non-ablative laser radiation and the whitening treatment.

In another aspect, the invention relates to a method for treating a dental hard tissue. The method can include the steps of: generating a laser beam using a laser source; applying a non-ablative laser radiation to a treatment surface of the hard dental tissue using an optic in optical communication with the laser source; and after the step of directing the non-ablative laser radiation, applying a whitening treatment to the hard dental tissue, wherein application of both the non-ablative laser radiation and the whitening treatment results in a Vita shade guide measurement of at least 6 times greater than a treatment surface without application of the non-ablative laser radiation and the whitening treatment.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a side cross-sectional schematic view of a laser based treatment system, according to various embodiments;

FIG. 2 is a chart providing example Delta E values of incisors for two groups, according to various embodiments;

FIG. 3 is a chart providing example hardnes values (KHN) for two groups, according to various embodiments; and

FIG. 4 is a chart providing example laser and treatment parameter values, according to various embodiments.

### DETAILED DESCRIPTION

Various embodiments of the present invention are directed to an improved laser-based treatment device that overcomes the shortcomings of conventional methods for teeth whitening. This disclosure will often describe the treatment system including a hand piece that delivers (i) laser pulses that heats the hard tissue to remove the carbonate and enhance remineralization / whitennig and (ii) a laser beam having a long working range (defined below), (iii) coolant (air, water) to an oral treatment region and (iv) delivery system for fluoiride or hydrogen peroxide application. In general, any suitable hard tissue region can be treated. Patterns for treating dental tissues are described in more detail in U.S. Patent Publication No. 20170319277, which is incorporated herein by reference in its entirety.

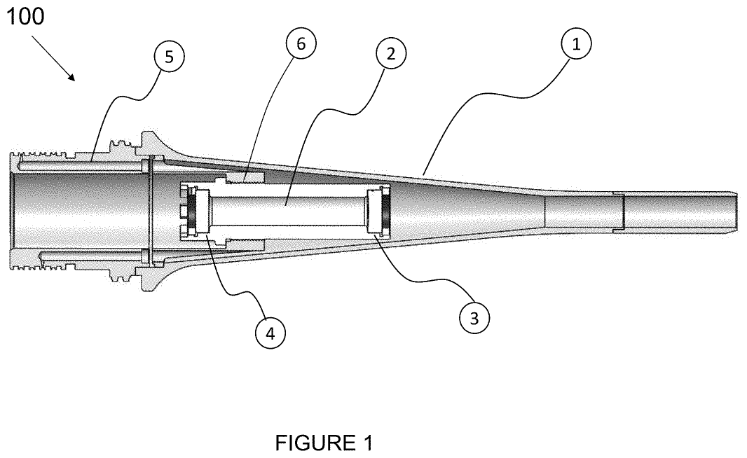

FIG. 1 is a side cross-sectional schematic view of a laser based treatment system including a handpiece and an optical cartridge, according to various embodiments. For some applications, a CO₂ laser source operating at a wavelength in a range of 9-11 μm (e.g., 9.3 μm), is desirable for such treatments. For example, CO₂ lasers can be delivered using a handpiece 1. As shown in FIG. 1, the handpiece 1 can have a configuration that can enable uniform treatment of various teeth with minimal change in technique sensitivuy. Some advantages of the laser therapy described herein over conventional methods include increased resistance to caries formation, improved fluoride/hydrogen peroxide uptake/absorption, on teeth.

In some embodiments, the laser is pulsed and/or scanned in a certain pattern to allow optimal surface modification without any damage to the pulp or strucutre. In some embodiments, the delivery of a collimated laser beam is achieved by a handpiece 1, which may be structured and designed to receive an optical cartridge 2. The optical cartridge 2 can include at least one optical lens to modulate a laser beam passing therethrough. For example, as shown in FIG. 1, the optical cartridge 2 can include an upstream optical lens 3 and a downstream optical lens 4. The optical cartridge 2 can be mounted to the handpiece 1 using known technique, e.g., with a threading 6. The ability to replace or remove the optical cartridge 2 allows switching the laser between treatment modes, such as an ablative mode to a non-ablative mode and vice versa. In addition, a conduit 5 in the handpiece may be used to deliver cooling fluids (e.g., air, water, and combinations thereof) to provide cooling of the tissue surface or a fluoride based fluid or a hydrogen based gel to enhance acid resitance and improve fluoride uptake and increase whitening. The conduit can be any suitable structure, e.g., a tubing, a lumen in the handpiece, etc.

In some embodiments, the optical cartridge 2 can provide a laser beam having a long working range. For example, the optical cartridge can produce a collimated and larger beam size (1 mm) and uniform over long working range (5-20 mm). As used herein the term “working range” refers to the distance along the length of the laser beam at which the laser beam has a fluence capable of treating the tissue. Conventional devices have a relatively short working range, typically focused tightly around the focal point of the laser beam, out of a desire to not waste any energy along the length of the laser.

In some embodiments, the laser treatment device of the present invention can tolerate a longer working range, so as to enable an operator to move their hand (and, corresponding, the laser beam), while still treating the treatment area. The working range is described in more detail with reference to the phrase “depth of treatment” (which can be interchanged with “working range”) in U.S. Patent Publication No. 20160143703, which is incorporated by reference herein in its entirety. In other words, in certain embodiments, the amount of energy on the target tissue does not change over a relatively long distance (e.g., more than 0.5 cm, more than 1 cm, more than 1.5 cm, more than 2 cm, more than 3 cm, more than 4 cm) to accommodate for hand movements and variability in the user's holding of the handpiece and to accommodate for human factors.

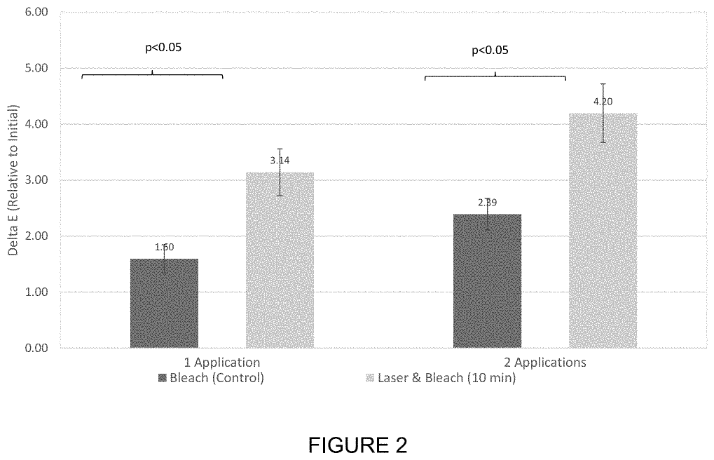

FIG. 2 is a chart providing example Delta E (or ΔE) measurement values of incisors for two groups, accodring to various embodiments. The two groups include a Control Group exposed to bleach only and a Lased Group exposed to laser irradiation followed by bleaching (or whitening) for 1 and 2 applications with 10 minutes for each of the applications. As shown in FIG. 2, the Laser Group exhibites a ΔE measurement of at least about 1.5, 1.6, 1.7, 1.8, 1.9, 2, 2.1, 2.2, 2.3, 2.4, 2.5, 2.6, 2.7, 2.8, 2.9, or 3 times greater than a treatment surface without application of the non-ablative laser radiation, after 1 and/or 2 applications. Thus, the Laser Group has whitening improvement over that of Control Group in at least half the treatment time.

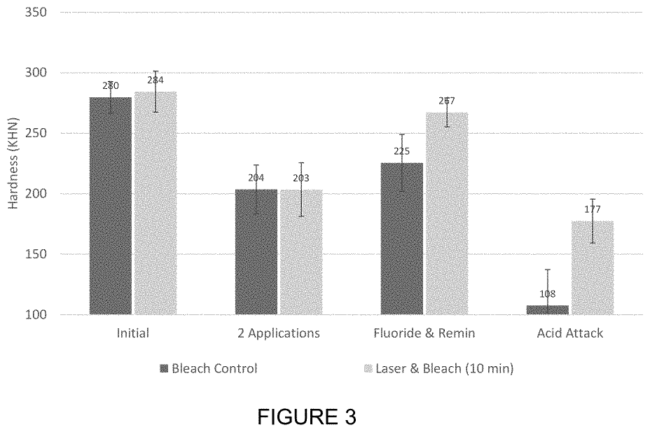

FIG. 3 is a chart providing example hardnes values (KHN) for two groups, according to various embodiments. The two groups include a Control Group exposed to bleaching for 2 sessions and a Treatment Group irradiated with a laser and followed by bleaching. Both groups are then treated with fluoride followed by a remoneralizaiotn and demineralization sessions. As shown in FIG. 3, the Treatment Group show: 1) increased hardness over the Control Group by at least about 18% after fluoride application and 2) at least about 64% inhibtion of demineralization.

In some embodiments, the effect on whitening using 9.3 um CO₂ laser is performed on a total of 30 human incisors in sound condition were obtained, cleaned, disinfected and transported in 0.1% thymol to prevent microbial growth before use (e.g., from Therametric Technologies, Inc., Nobelsville, Ind.). The samples were mounted in Durabase hard chairside reline (e.g., from Reliance Dental Mfg, Alsip, Ill.) with the labial surface up, and with the roots covered. The samples were then polished in a standard manner using a rotary polisher and pumice paste. The samples were randomly divided into 3 groups: group 1 was a control group with only bleach (Opalescence Boost, Opalescence, South Jordan, Utah); group 2 samples were irradiated with the laser, followed by two applications of bleach for 10 minutes each; group 3 samples were irradiated with the laser, followed by two applications of bleach for 20 minutes each. Samples were left in a 1:3 slurry of toothpaste (e.g., from Crest cavity protection, Proctor and Gamble, Inc., Cincinnati, Ohio) to distilled water. The samples were left in a Tris-HCl pH 7.1 remineralization buffer (e.g., from Boston Bioproducts, Ashland, Mass.) for a total of 10 days. At 4 days, the samples were subject to an acid challenge by exposing them to a citric acid buffer of pH 3.6 for 6 minutes before being returned to the remineralization solution.

### Spectroscopy Whitening Measurements

The effect of tooth whitening was measured by both a spectroscopy method and manual method matching tooth shade to a shade guide. Spectroscopy was done using a color/shade measurement device (MHT Spectroshade Micro, Oxnard Calif.), with tooth surfaces wet and the mounted samples placed in a black box. The device outputs brightness (L), red-green axis (a), and blue-yellow axis (b) values for each measurement. These values are combined into one value, ΔE, ^{3,4} which represents the overall color change between a pair of measurements, calculated by: ΔE=\sqrt{(L2−L1)²+(a2−a1)²+(b2−b1)²}, where L represents lightness, a represents redness-greenness of the color, and b represents yellowness-blueness of the color. Higher ΔE values indicate a greater effect of whitening, and should be associated with an increase in L, and a decrease in a and b. These values were calculated at each step of the experiment for the 3 groups.

### Shade Guide Whitening Measurements

A Vita Shade guide was used to manually assign shade values to each sample. The shade guide has 16 shades of samples, ranked from 1-16, with 1 being the brightest. Vita shade guide measurements were performed at the start of the experiment, and before and after the final stain. Teeth were selected initially if they had shades ≥9, or at least an A3 on the shade guide.

### Microhardness Measurements

To prepare for microhardness measurements, a 3-5 mm diameter area of the incisor surface was serially polished with 800, 1200 and 1-μm diamond grit. A microhardness tester Matsuzawa Seiki DMH-2, (Matsuzawa, Akita Pref, Japan) was used with a Knoop diamond tip and a load of 50 g, with a 10 s dwell time. 5 indents were taken on each sample at the start of the experiment, after bleaching, after remineralization, and after the acid challenge. Indent lengths were measured in a digital microscope (RH-2000, Hirox-USA, Inc., Hackensack, N.J.) at 1000× magnification. Indent lengths were converted to knoop hardness (KHN) using the following formula:

$$KHN = \frac{g}{0.0703 * {I}^{2}} ,$$

where g is the load in grams, and l is the indent length in millimeters.

Irradiation of hard tissue followed by application of bleach improved the whitening effect by a factor of 2 in only half the treatment time over the Control group as shown in FIG. 2. The ΔE values are those taken relative to initia lor before treatment of teeth with either bleach or laser irradiation. ΔE increased by a factor of 3.14 for the Lased Group after 10 min session of bleach ad to 4.2 after a second 20 min application. On the other hand, the whitening of the Control Group exhibited a factor 1.5 and 2.39 after 10 min and 20 min of bleach application, repsepctively.

Measurement of surface hardness represented by (KHN) in FIG. 3 characterize strength or weakning of the tooth after bleach treatment. After 2 sessions (10 minutes each) of bleach appplcaiton, both the Control and Treatment Groups lost half of their original values. However, after remineralization cycle that involves fluoride, the surface hardness of the Treatment Group improved by 18% over that of the Control Group. More importantly, the Treatment Group inhibited demineralization of the surface tooth by 64% over the Control Group.

In some variations, radiation emitted by a laser source may be transmitted through the hand piece accompanied by fluids. The cooling is carried through fluid tubings 5 that run along the handpiece 1 and circumvent the optical cartridge 2. The cooling might be useful to reduce any un-intentional heating of the tissue.

In some embodiments of the invention, the fluid tubings 5 carries fluoride/hydrogen peroxide based fluids to improve the effectiveness of the treatment after the laser irradaiton of hard tissue that is performed through the handpiece.

In certain embodiments, the CO₂ laser is accompanied with a marking beam (e.g., green in color) that serves as a guidance of the location of the laser beam on the target tissue. In other embodiments, the irradiation of the laser may consist of a pattern. A visual or sonar feedback can be integrated within the system to indicate to the user the need to move to a new target area. A visual feedback to move for a new target area can include a stationary guidance beam (e.g., a green point can be seen on the tissue). For example, while the tissue is being exposed to the laser, a pattern is displayed on the tissue. When enough dose of energy has been delivered in a pattern, the laser can stop scanning and a point object can being projected on the target tissue. Alternatively, a sonar feedback can include a sound emerging from the system when the sequence of patterns or energy dose is delivered.

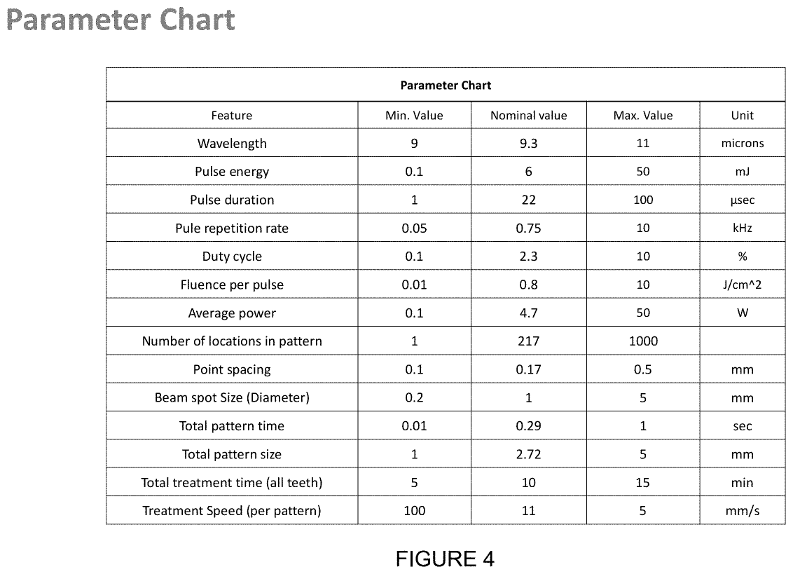

FIG. 4 is a chart providing example laser and treatment parameter values, according to various embodiments. In various embodiments, laser parameters (e.g., power, repetition rate, pulse duration, and/or laser beam overlap) may be selected to optimize efficiency to remove carbonate without damaging the material (i.e., optical cartridge 2) itself and to contact tissue without damage. In some embodiments, the laser source may be spatially scanned to provide different pulse energies at different locations, as will be appreciated by those skilled in the art.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the chart shown in FIG. 4), is contemplated and expressly supported herein, subject to the number of significant digits expressed in each particular range. FIG. 4 also provides express support for the ranges between minimal and nominal, nominal and maximum, and minimum and maximum for each parameter listed.

Having described herein illustrative embodiments of the present invention, persons of ordinary skill in the art will appreciate various other features and advantages of the invention apart from those specifically described above. It should therefore be understood that the foregoing is only illustrative of the principles of the invention, and that various modifications and additions can be made by those skilled in the art without departing from the spirit and scope of the invention. Accordingly, the appended claims shall not be limited by the particular features that have been shown and described but shall be construed also to cover any obvious modifications and equivalents thereof.

What is claimed is:

1. A system for treating a dental hard tissue, comprising:
a laser source for generating a laser beam;
at least one optic in optical communication with the laser source and adapted to define and direct a non-ablative laser radiation to a treatment surface of the dental hard tissue; and
a conduit adapted to apply a whitening treatment to the treatment surface, wherein application of both the non-ablative laser radiation and the whitening treatment results in a ΔE measurement of at least 2.5 times greater than a treatment surface without application of the non-ablative laser radiation.

2. The system of claim 1, wherein the laser beam comprise a wavelength in a range from 9 μm to 11 μm.

3. The system of claim 1, wherein the at least one optic comprises a galvanometer.

4. The system of claim 3, wherein the at least one optic further comprises a turning mirror.

5. The system of claim 1, wherein the non-ablative laser radiation comprises a fluence profile at a focus having:
a maximum local fluence less than an upper threshold fluence, the upper threshold fluence defined as a minimum fluence that causes a surface modification of the dental hard tissue, and
at least one other local fluence greater than a lower threshold fluence, the lower threshold fluence defined as a fluence that causes at least one of (i) acid dissolution and (ii) decrease in an amount of surface carbonate.

6. The system of claim 5, wherein the surface modification comprises at least one of melting and ablation.

7. The system of claim 1, wherein the whitening treatment comprises at least one of bleach and hydrogen peroxide.

8. The system of claim 1, wherein application of both the non-ablative laser radiation and the whitening treatment further results in a microhardness measurement of at least 18% greater than the treatment surface without application of the non-ablative laser radiation, after bleach applications and a remineralization cycle comprising fluoride.

9. The system of claim 8, wherein application of both the non-ablative laser radiation and the whitening treatment further results in an inhibited demineralization of at least 64% greater than the treatment surface without application of the non-ablative laser radiation.

10. The system of claim 8, wherein application of both the non-ablative laser radiation and the whitening treatment further results in a Vita shade guide measurement of at least 6 times greater than the treatment surface without application of the non-ablative laser radiation.

11. The system of claim 1, wherein application of both the non-ablative laser radiation and the whitening treatment further results in a Vita shade guide measurement of at least 6 times greater than the treatment surface without application of the non-ablative laser radiation and the whitening treatment.

12. A system for treating a dental hard tissue, comprising:
a laser source for generating a laser beam;
at least one optic in optical communication with the laser source and adapted to define and direct a non-ablative laser radiation to a treatment surface of the hard dental tissue; and
a conduit adapted to apply a whitening treatment to the treatment surface, wherein application of both the non-ablative laser radiation and the whitening treatment results in a microhardness measurement of at least 18% greater than a treatment surface without application of the non-ablative laser radiation.

13. A system for treating a dental hard tissue, comprising:
a laser source for generating a laser beam;
at least one optic in optical communication with the laser source and adapted to define and direct a non-ablative laser radiation to a treatment surface of the hard dental tissue; and
a conduit adapted to apply a whitening treatment to the treatment surface, wherein application of both the non-ablative laser radiation and the whitening treatment results in a Vita shade guide measurement of at least 6 times greater than a treatment surface without application of the non-ablative laser radiation and the whitening treatment.

14.-16. (canceled)
